(19) United States
(12) Patent Application Publication
(10) Pub. No.: US 2021/0393743 A1
(43) Pub. Date: Dec. 23, 2021

(54) Use of IL13 for prevention and treatment of COVID19

(71) Applicant: Ketan Desai, Easton, PA (US)

(72) Inventor: Ketan Desai, Easton, PA (US)

(21) Appl. No.: 17/334,746

(22) Filed: May 30, 2021

Related U.S. Application Data
(60) Provisional application No. 63/041,958, filed on Jun. 21, 2020.

(51) Int. Cl. A61K 38/20; A61K 47/69; A61K 47/62; A61K 45/06; A61K 35/16; A61P 31/14

(52) U.S. Cl. CPC A61K 38/2086; A61K 47/6951; A61K 47/6911; A61K 48/00; A61K 45/06; A61K 35/16; A61P 31/14; A61K 47/62

(57) ABSTRACT

A method of use for prevention and treatment of COVID-19 using IL13 is described.

This application claims priority to U.S. Provisional Application No. 63/041,958, the contents of which are hereby incorporated by reference in its entirety for all purposes

### BACKGROUND OF THE INVENTION

COVID-19 is a relatively new coronavirus infection first detected in 2019. It is now a pandeic for which no clinical cure exits. It has been observed that asthmatics who suffer from COVID-19 do better than control patients. It is postulated that Interleukin 13, one of the interleukins important in that pathogenesis of asthma is in fact protective against COVID 19. It does so by decreasing the ACE2 receptor on surface of the cells, which SARS-COV2, the causative virus, binds. It also decreases the hyper inflammatory response, thus dampening the cytokine storm that causes fatality in the terminal stages. It can therefore be used as a therapeutic to treat COVID-19.

### SUMMARY OF THE INVENTION

This invention describes the science behind the use of IL-13 to treat COVID-19, chemistry of IL-13, pre-clinical data supporting such use in humans, and relevant references

### DETAILED DESCRIPTION

In December of 2019, a novel Coronavirus, SARS-CoV-2, emerged in China and has gone on to trigger a global pandemic of Coronavirus Disease 2019 (COVID-19), the respiratory illness caused by this virus¹. While most individuals with COVID-19 experience mild cold symptoms (cough and fever), some develop more severe disease including pneumonia, which often necessitates mechanical ventilation. An estimated 5.7% of COVID-19 illnesses are fatal. Enhanced risk of poor outcomes for COVID-19 has been associated with a number of factors including advanced age, male gender, and underlying cardiovascular and respiratory conditions. However, one disease state leads to a favorable outcome in patients with COVID-19. Patients with asthma have been found to have better mortality and morbidity than others when infected with SARS-CoV-2 ²⁻⁶

One factor that may underlie variation in clinical outcomes of COVID-19 is the extent of gene expression in the airway of the SARS-CoV-2 entry receptor, ACE2. Expression of these genes and their associated programs in the nasal airway epithelium is of particular interest given that the nasal epithelium is the primary site of infection for upper airway respiratory viruses, including coronaviruses, and acts as the gateway through which upper airway infections can spread into the lung. The airway epithelium is composed of multiple resident cell types (e.g., mucus secretory, ciliated, basal stem cells, and rare epithelial cell types) interdigitated with immune cells (e.g. T cells, mast cells, macrophages), and the relative abundance of these cell types in the epithelium can greatly influence the expression of particular genes, including ACE2. Furthermore, since the airway epithelium acts as a sentinel for the entire respiratory system, its cellular composition, along with its transcriptional and functional characteristics, are significantly shaped by interaction with environmental stimuli. These stimuli may be inhaled (e.g., cigarette smoke, allergens, microorganisms) or endogenous, such as when signaling molecules are produced by airway immune cells present during different disease states. One such disease state is allergic airway inflammation caused by type 2 (T2) cytokines (IL-4, IL-5, IL-13), which is common in both children and adults and has been associated with the development of asthma. T2 cytokines are known to greatly modify gene expression in the airway epithelium, both through transcriptional changes within cells and epithelial remodeling in the form of mucus metaplasia ⁷⁻⁸

As mentioned, there is tremendous population variation in upper airway expression of the ACE2 receptor for SARS-CoV-2 and this could drive infection susceptibility and disease severity. Network and eQTL analysis of nasal airway epithelial transcriptome data from a large cohort of healthy and asthmatic children aged 8-21 years showed a dramatic influence of T2 cytokine-driven downregulation of ACE2 ⁷⁻⁸

Airway inflammation caused by type 2 cytokine production from infiltrating immune cells plays a prominent role in the control of cellular composition, expression, and thus biology of the airway epithelium. T2 inflammation induced with IL-13 stimulation precipitated a dramatic reduction in levels of epithelial ACE2. Germane to this question, a recent study of 85 fatal COVID-19 subjects found that 81.2% of them exhibited very low levels of blood eosinophil levels. Blood eosinophil levels are a strong, well-known predictor of airway T2 inflammation and were strongly correlated with T2 status.

Together, these studies provisionally suggest that T2 inflammation may predispose individuals to experience better COVID-19 outcomes through a decrease in airway levels of ACE2 ⁷⁻⁸

IL13 is an immunoregulatory cytokine produced primarily by activated Th2 cells ¹². IL-13 is involved in several stages of B-cell maturation and differentiation. It up-regulates CD23 and MEW class II expression, and promotes IgE isotype switching of B cells. This cytokine down-regulates macrophage activity, thereby inhibits the production of pro-inflammatory cytokines and chemokines seen in COVID-19 cytokine storm. It decreases T1 phenotype and interleukins/cytokines associated with it including IL-6, IL-2, TNF and gamma interferon. This cytokine is found to be critical to the pathogenesis of allergen-induced asthma but operates through mechanisms independent of IgE and eosinophils. This gene, IL3, ILS, IL4, and CSF2 form a cytokine gene cluster on chromosome 5q, with this gene particularly close to IL4.

Given that asthmatics have a better outcome with IL-13 being the key mediator in switching the T1 phenotype to the T2 phenotype, and decreasing the levels of ACE-2, it is proposed that IL-13 be used in the treatment of COVID-19.

Chemistry:

Interleukin-13 Human produced in E. Coli is a single, non-glycosylated polypeptide chain containing 112 amino acids and a molecular mass of 12 kDa.

The IL-13 is purified by chromatography.

Source

Escherichia Coli.

Physical appearance

Sterile Filtered White lyophilized (freeze-dried) powder.

Formulation

The protein (1 mg/ml) was lyophilized with 1×PBS pH-7.2 & 5% trehalose.

Solubility

It is recommended to reconstitute the lyophilized Interleukin 13 in sterile 18MΩ-cm H2O not less than 100 μg/ml, which can then be further diluted to other aqueous solutions.

Stability

Lyophilized Interleukin-13 although stable at room temperature for 3 weeks, should be stored desiccated below −18° C. Upon reconstitution IL13 should be stored at 4° C. between 2-7 days and for future use below −18° C.

For long term storage it is recommended to add a carrier protein (0.1% HSA or BSA).

Purity

Greater than 95% as determined by:

(a) Analysis by RP-HPLC.

(b) Analysis by SDS-PAGE.

Amino Acid Sequence

GPVPPSTALRELIEELVNITQNQKAPLCNGSMVWSINLTAGMYCAALESLINVS GCSAIEKTQRMLSGFCPHKVSAGQF SSLHVRDTKIEVAQFVKDLLLHLKKLFRE GRFN.

Biological Activity

The ED50 was determined by the dose dependent prolifiration of TF-1 cells and was found to be <1 ng/ml, corresponding to a specific activity of >1×10⁶units/mg.

Protein Content

Protein quantitation was carried out by two independent methods:

1. UV spectroscopy at 280 nm using the absorbency value of 0.57 as the extinction coefficient for a 0.1% (1 mg/ml) solution. This value is calculated by the PC GENE computer analysis program of protein sequences (IntelliGenetics).

2. Analysis by RP-HPLC, using a calibrated solution of IL-13 as a Reference Standard.

Pre-Clinical Data:

To test the effect of IL-13 in COVID-19, we utilized a K18-hACE2 transgenic mouse model of COVID-19 9-11. In this model mice progress to severe disease starting at day five post-infection (pi) with SARS-CoV-2.

To directly test whether IL-13 decreases SARS-CoV-2 infection, we administered intraperitoneal (i.p.) injections of IL-13 or saline on days 0, 2 and 4 post infection. Infected mice receiving IL-13 had significantly reduced symptoms as measured by clinical scores, weight loss, and mortality.

Mice were infected on day 0 with 5×10³ PFU of SARS-CoV-2 and administered 0.1 μg of IL-13 or normal saline intraperitoneally on days 0, 2, and 4. Clinical scoring was measured by weight loss (0-5), posture and appearance of fur (piloerection) (0-2), activity (0-3) and eye closure (0-2). Weight loss was measured by weighing on days 7 post infection. Mortality was measured at day 7

Materials and Methods

Virus and Cell Lines:

SARS-Related Coronavirus 2 (SARS-CoV-2), isolate Hong Kong/VM20001061/2020 (NR-was obtained from the Biodefense and Emerging Infections Research Resources Repository (BEI Resources), National Institute of Allergy and Infectious Diseases (NIAID), National Institutes of Health (NIH). Virus was propagated in Vero C1008, Clone E6 (ATCC CRL-1586) cells cultured in Dulbecco's Modified Eagle's Medium (DMEM, Gibco 11995040) supplemented with 10% fetal bovine serum (FBS) and grown at 37° C., 5% CO2. Initial viral stocks were used to infect Vero E6 cells, generating passage 1 (P1) stocks. These P1 stocks were then used to infect additional Vero E6 cells, generating passage 2 (P2) stocks, which were used for all experiments.

Viral Propagation:

Vero E6 cells grown to 90% confluency in T75 tissue culture flasks (Thermo Scientific) were infected with SARS-CoV-2 at a multiplicity of infection of 0.025 in serum-free DMEM. Vero E6 cells were incubated with virus for two hours at 37° C., 5% CO2, after which the virus was removed, media was replaced with DMEM supplemented with 10% FBS, and flasks were incubated at 37° C., 5% CO2. After two days, infected flasks showed significant cytopathic effects, with >50% of cells unattached. Cell supernatants were collected, filtered through a 0.22 μm filter (Millipore, SLGP003RS), and centrifuged at 300×g for ten minutes at 4° C. Cell supernatants were divided into cryogenic vials (Corning, 430487) as viral stocks and stored at 80° C. until use.

Challenge: 8-16 week-old -male Tg (K18-hACE2) 2Prlmn (Jackson Laboratories) (Moreau et al., 2020) mice were challenged with 5000 plaque forming units (PFUs) of SARS-CoV-2 in 50 μL by an intranasal route under ketamine/xylazine sedation. Mice were followed daily for clinical symptoms, which included weight loss (0-5), activity (0-3), fur appearance and posture (0-2), and eye closure (0-2). Mice were given 0.1 μg of IL-13 or saline administered on day 0, 2, and 4 post infection.

Statistical Methods:

For clinical scores, weight loss, a two-tailed Student's t test was used to determined statistical significance. Response differences between groups (e.g., infected vs. uninfected) were evaluated in the mixed-effects model to account for within-individual correlation, and distributions were log transformed where appropriate. P value<0.05 was considered significant.

Results:

Clinical Scores:

| Day | Saline | IL-13 |
|---|---|---|
| 1 | 0 | 0 |
| 7 | 4 | 11 |

Weight Loss:

| Day | Saline (% starting weight) | IL13 (% starting weight) |
|---|---|---|
| 1 | 100 | 100 |
| 7 | 85 | 94 |

Survival %:

| Day | Saline | IL13 |
|---|---|---|
| 1 | 100 | 100 |
| 7 | 25 | 75 |

P=0.0056

As the data shows, IL13 administration resulted in improved survival, decreased weight loss, and improved clinical scores in mice. It is reasonable to extrapolate that these results will be replicated in humans with COVID-19, and thus IL-13 is a therapeutic option for patients suffering from SARS-COV2 infection

### REFERENCES

1. 2019 Novel Coronavirus (2019-nCov) outbreak: A new challenge. Lupia T et al. Journal of Global Antimicrobial Resistance. Vol 21 June 2020. Pages 22-27.

2. Type 2 and interferon inflammation regulate SARS-CoV-2 entry factor expression in the airway epithelium. Sajuthi, S. P et al, Nature Communications volume 11, Article number: 5139 (2020)

3. Presence of co-morbid asthma in Covid 19 patients. Butler, M. W et al. J Allergy and Clin Immunology Vol 146, No 2. (2020)

4. Risk factors for severity and mortality in adult COVID-19 inpatients in Wuhan. Xiochen Li, et al. J Allergy and Clin Immunology Vol 146, No 1 (2020)

5. Comorbidity and its impact on 1590 patients with Covid-19 in China: A nationwide analysis. Guan W et al. European Respiratory Journal 2020, 55: 2000547 DOI: 10.1183/13993003.00547-2020

6. COVID-19 Susceptibility in Bronchial Asthma. Green I. Et al. The Journal of Allergy and Clinical Immunology: In Practice. Available online 24 Nov. 2020

7. Covid -19 susceptibility in bronchial asthma. Green, Ila et al. Journal of Allergy and Clinical Immunolgy in Practice. Volume 9, Issue 2, February 2021, Pages 684-692

8. Type 2 and interferon inflammation strongly regulate SARS-CoV-2 related gene 3 expression in the airway epithelium. Sajuthi S. P. Et al. Nature Communications. 2020 Oct. 12; 11(1):5139

9. Moreau, G. B., S. L. Burgess, J. M. Sturek, A. N. Donlan, W. A. Petri, and B. J. Mann. 2020. Evaluation of K18-hACE2 Mice as a Model of SARS-CoV-2 Infection. Am. J. Trop. Med. Hyg. 103:1215-1219. doi:10.4269/ajtmh.20-0762.

10. Rathnasinghe, R., S. Strohmeier, F. Amanat, V. L. Gillespie, F. Krammer, A. García-Sastre, L. Coughlan, M. Schotsaert, and M. B. Uccellini. 2020. Comparison of transgenic and adenovirus hACE2 mouse models for SARS-CoV-2 infection. Emerg. Microbes Infect. 9:2433-2445. doi:10.1080/22221751.2020.1838955.

11. Winkler, E. S., A. L. Bailey, N. M. Kafai, S. Nair, B. T. McCune, J. Yu, J. M. Fox, R. E. Chen, J. T. Earnest, S. P. Keeler, J. H. Ritter, L.-I. Kang, S. Dort, A. Robichaud, R. Head, M. J. Holtzman, and M. S. Diamond. 2020. Publisher Correction: SARS-CoV-2 infection of human ACE2-transgenic mice causes severe lung inflammation and impaired function. 10 Nat. Immunol. 21:1470-1470. doi:10.1038/s41590-020-0794-2.

12. The Intriguing Role of Interleukin 13 in the Pathophysiology of Asthma. Marone Giancarlo, Granata Francescopaolo, Pucino Valentina, Pecoraro Antonio, Heffler Enrico, Loffredo Stefania, Scadding Guy W., Varricchi Gilda. Frontiers in Pharmacology. VOLUME=10. 2019: 1387

What is claimed:

1. Method of use of IL13 to treat COVID-19.

2. Method of use as described in claim 1 where IL13 is given in therapeutic or a post exposure prophylactic setting.

3. Method of use as described in claim 1 when given as a therapeutic to mild, moderate, severe, critical COVID patients and those suffering from long term sequelae of COVID-19 aka long haulers.

4. Method of use as described in claim 1 where IL13 is given orally, subcuteneously, intra dermally, intramuscularly, intravenously, intraperitoneally, rectally, transdermally, sublingually, or by inhalation.

5. Method of use as described in claim 1 where IL13 is given for the duration of illness or potential exposure or for part of the illness.

6. Method of use as described in claim 1 where IL13 is given alone or in combination with other therapies, including but not limited to biologics, antibodies, anti-virals, plasma, or vaccines.

7. Method of use as described in claim 1 where IL13 is given as a peptide or as a gene therapy.

8. Method of use as described in claim 1 where IL13 is given as a whole length molecule or fragment.

9. Method of use as described in claim 1 where IL13 is given alone or in a complex with including but not limited to liposomes, carrier proteins, cyclodextrins, etc.

10. Method of use as described in claim 1 where IL13 is given in doses ranging from 1 picogram to 100 milligram per administration.
